(19) United States
(12) Patent Application Publication
(10) Pub. No.: US 2024/0350808 A1
(43) Pub. Date: Oct. 24, 2024

(54) SYSTEMS AND METHODS FOR AN IMPLANTABLE DEVICE

(71) Applicant: GALVANI BIOELECTRONICS LIMITED, Brentford, Middlesex (GB)

(72) Inventors: David Karl Lee PETERSON, Brentford, Middlesex (GB); Kevin KWONG, Brentford, Middlesex (GB); Gerald Edwin HUNSBERGER, Brentford, Middlesex (GB); Jason MIRANDA, Brentford, Middlesex (GB); Isha GUPTA, Brentford, Middlesex (GB); Paul MATTEUCCI, Brentford, Middlesex (GB); Daniel CHEW, Brentford, Middlesex (GB)

(73) Assignee: GALVANI BIOELECTRONICS LIMITED, Brentford, Middlesex (GB)

(21) Appl. No.: 18/685,989

(22) PCT Filed: Aug. 26, 2022
(86) PCT No.: PCT/GB2022/052205

§ 371 Date: Feb. 23, 2024

Related U.S. Application Data
(60) Provisional application No. 63/260,632, filed on Aug. 27, 2021.

(51) Int. Cl. A61N 1/36; A61N 1/05; A61N 1/08; A61N 1/375

(52) U.S. Cl. CPC A61N 1/3614; A61N 1/0556; A61N 1/36139; A61N 1/36142; A61N 1/36146; A61N 1/375; A61N 2001/083

(57) ABSTRACT

Systems for applying electrical stimulation to a nerve and determining a change in measured impedance or sensed electrical fields that is indicative of an electrical event, are disclosed herein. The system comprises an implantable housing comprising a signal generator, an implantable neural interface, an impedance measuring circuit or an electrical field sensing circuit, and processing circuitry. Methods for determining the presence of an electrical event in a system for applying electrical stimulation to a nerve, are also disclosed herein. The method comprises periodically sensing an impedance in an electrical pathway and determining an electrical event in the electrical pathway by identifying a change in the sensed impedance.

### FIELD

The present invention relates to systems and methods for applying electrical stimulation to a nerve, and in particular to taking impedance measurements via electrical pathways in an implantable system into order to determine changes that are indicative of an electrical event.

### BACKGROUND

US2012303096 discloses methods and systems for automatically detecting an impedance of one or more electrodes in a cochlear implant system. The methods and systems include providing an implantable cochlear stimulator coupled to one or more electrodes, generating an electrical stimulation current with the implantable cochlear stimulator in accordance with stimulation parameters, automatically detecting an impedance of at least one of the electrodes in accordance with a predefined schedule, and performing a predefined action in accordance with the detected impedance.

US2012300953 discloses an implantable hearing prosthesis, comprising an integrated integrity system. The integrity system is configured to measure one or more electrical characteristics of at least one component of the prosthesis, to evaluate the integrity of the prosthesis based on the measurements, and to perform at least one diagnostic operation based on the evaluation.

US2005256547 discloses methods for failure recovery in a cardiac rhythm management system and apparatus capable of carrying out the methods. The methods include applying a first pacing therapy using one or more leads. The methods further include detecting a failure condition on one or more of the leads, wherein the failure condition prohibits or frustrates application of the first pacing therapy. The methods still further include applying a second pacing therapy using one or more of the leads subsequent to detecting the failure condition. The second pacing therapy is preferably chosen such that the detected failure does not interfere with the second pacing therapy. The second pacing therapy may be applied for only one cardiac cycle. The second pacing therapy may further be applied continuously until the failure condition is resolved, or it may be latched such that physician intervention is required to resume the first pacing therapy.

US2014276151 discloses an implantable system that includes terminals, a pulse generator, a sensing circuit, separate signal processing channels, and first, second and third multiplexers. The terminals are connected to electrodes via conductors of leads. Different subsets of the electrodes are used to define different electrical pulse delivery vectors, and different subsets of the electrodes are used to define different sensing vectors. The pulse generator produces electrical pulses, and the sensing circuit senses a signal indicative of an impedance associated with a selected sensing vector. The first multiplexer selectively connects outputs of the pulse generator to a selected one of the different electrical pulse delivery vectors at a time. The second multiplexer selectively connect inputs of the sensing circuit to a selected one of the different sensing vectors at a time. The third multiplexer selectively connects an output of the sensing circuit to one of the plurality of separate signal processing channels at a time.

US2006247706 discloses diverse methods and apparatuses for in vivo monitoring, detecting and/or predicting potential failure modes or deleterious trends of chronically implanted medical electrical leads prior to actual failure of said leads. Certain disclosed embodiments involve applying a relatively increased data sampling rate at various time intervals (e.g., periodically, randomly, and/or manually-triggered and the like) prior to actual detection of a deleterious trend, to thereby increase the probability of detecting one or more parameters indicative of a potential lead performance issue. At least some of the parameters are utilized because they are not typically reliably detected at relatively lower data sampling rates. In addition to an initial relatively increased data sampling rate, certain embodiments provide for adjusting the sampling rate and storing and/or adjusting data pattern template-based data to execute pattern related triggers so that additional information regarding a medical electrical lead can be obtained.

The present invention addresses deficiencies of the prior art, as they pertain to systems and methods for applying electrical stimulation to a nerve.

### SUMMARY OF INVENTION

In one aspect, a system for applying electrical stimulation to a nerve includes an implantable housing that includes a signal generator. The housing includes an electrically conductive portion for contacting tissue, in use (in other words, the electrically conductive portion is configured to contact the tissue). The system comprises an implantable neural interface for being in signaling contact with one or more nerves, the implantable neural interface includes at least a first electrode electrically coupled to the signal generator for applying a signal generated by the signal generator to the one or more nerves. The system further comprises an impedance measuring circuit electrically coupled to the electrically conductive portion of the implantable housing and the first electrode of the implantable neural interface and configured to measure an impedance of an electrical pathway therebetween. The system further comprises processing circuitry configured to periodically cause the impedance measuring circuit to measure impedances, the processing circuitry configured to determine a change in measured impedance that is indicative of an electrical event in a component in the electrical pathway that includes the first electrode. The system may be fully implantable, or alternatively the system may comprise implantable components and components which are not implanted. In the case where the system is fully implantable, components may be implantable in different locations. The processing circuitry may comprise one or more subcircuits, each subcircuit performing one or more of the functions assigned to the processing circuitry. The processing circuitry subcircuits may be provided entirely on one or more implantable parts of the system. Alternatively, the processing circuitry subcircuits may be provided entirely on one or more external parts of the system. Alternatively, the processing circuitry subcircuits may be distributed between one or more implantable parts of the system and one or more external parts of the system. The functions of the processing circuitry may be completely performed by one or more implantable components, the functions may be completely performed by one or more external components, or some of the functions may be performed by implantable components and some may be performed by external components. For example, the impedance data collected in the IPG may be transferred to or shared with, by wired or wireless communication, an external remote device such as a clinician programmer and/or patient remote such that the impedance data processing to determine a change in measured impedance data that is indicative of an electrical event may be carried out in the external remote device. The clinician programmer and/or patient remote, or an equivalent external remote device, may have cloud connectivity such that the impedance data are processed in the cloud. Any appropriate actions based on the analysis may be prescribed to the IPG through connectivity by wired or wireless communication.

An electrical event may be an electrical defect, such as an electrical discontinuity, a break in an electrical signal path, or a short circuit. However, other electrical events, are contemplated that allow for the performance of the implanted device to be monitored. Said performance may refer to an internal performance of the device itself, wherein impedance monitoring and actions are taken in response to the impedance monitoring as discussed here to maintain the system within a desired level of performance (e.g. within a desired level of electrical connectivity).

The implantable neural interface may further include a second electrode electrically coupled to the signal generator. The impedance measuring circuit may be electrically coupled to the second electrode and configured to measure an impedance of an electrical pathway between the electrically conductive portion of the implantable housing and the second electrode and/or of an electrical pathway between the first electrode and the second electrode. The processing circuitry is further configured to determine a change in measured impedance that is indicative of an electrical event in the electrical pathway that includes the second electrode.

A first electrical pathway may comprise the first electrode and the electrically conductive portion of the implantable housing. A second electrical pathway may comprise the second electrode and the electrically conductive portion of the implantable housing. The processing circuitry may be configured to detect whether a measured impedance value in one or both of these pathways rises above a first absolute threshold value or falls below a second absolute threshold value. The first absolute threshold value may be higher than the second absolute threshold value.

The measured impedances in the first and second electrical pathways may be substantially the same. If, however, an electrical event occurs in one of the electrical pathways, the measured impedance of that pathway may vary relative to that of the other electrical pathway. The processing circuitry may be configured to detect a difference in the measured impedance values in the first and the second electrical pathways (e.g. a change of the measured impedance value in one electrical pathway against the measured impedance values of the other electrical pathway). The difference in measured impedance between the first and second electrical pathways may be expressed as a percentage difference. This may be calculated by determining the difference in impedance between the pathways and expressing this difference as a percentage of the impedance of the pathways when absent the occurrence of the electrical event. If the difference (or the change) in the first and the second electrical pathways is between a predetermined difference range or above a predetermined threshold, the processing circuitry may determine and/or indicate an electrical event. In one embodiment, an electrical event may be indicated where the difference between the impedances of the electrical pathways, or the change in impedance of one electrical pathway compared with the other is between 20%-100%, 30%-90%, 40%-80%, 50%-70% or 50%-65% of the impedance of the electrical pathway that has not experienced the change. However, these lower and upper bounds are purely exemplary, and other bound values fall within the scope of the invention, for example the upper bound may be in excess of 100%. In one embodiment, an electrical event may be indicated where the difference between the impedances of the electrical pathways, or the change in impedance of one electrical pathway compared with the other is above 20%, 30%, 40%, 50%, 65%, 80%, 90%, or 100% of the impedance of the electrical pathway that has not experienced the change (or not as significant a change or a smaller change). Again however, these values are purely exemplary and other predetermined difference values fall within the scope of the invention, for example a predetermined difference value may be in excess of 100%. By way of an example, if the impedance of the first and second electrical pathways starts at a first impedance A, and the impedance of the second electrical pathway changes to a second impedance B, then a percentage change, Δ%, may be calculated based on the first impedance, and calculated as Δ%=(B−A)Ω/AΩ.

The first electrode and the second electrode may each comprise an array of electrodes. The processing circuitry may be configured to detect a change in the impedance of the electrical pathways using rolling average of the impedance data and/or z-score measured in terms of deviation from the average of each electrical pathway. The z-score may be calculated using Z=(x−μ)/σ, where x is an observed impedance value, μ is a rolling average of the sample, and σ is the standard deviation of the sample. The sample comprises impedance data of the electrical pathway. The z-score may be used to detect any abrupt change in the rolling average. The z-score may act as a threshold to trigger detection of an electrical event. Separate rolling average may be monitored for the first and the second electrical pathways respectively. A change of more than 25% in an absolute value or deviation from the rolling average or deviation from the baseline impedance value may indicate an electrical event. A rolling average of the difference between the first and the second electrical pathway may also be monitored.

The system may further include a communication subsystem configured to couple to a remote device and a control system operatively coupled to the signal generator and configured to cause the communication subsystem to transmit an alert to the remote device in response to a determination by the processing circuitry that the change in measured impedance is indicative of an electrical event.

The system may also include a control system operatively coupled to the signal generator and configured to modify the signal generated by the generator in response to a determination by the processing circuitry that the change in measured impedance is indicative of an electrical event. The control system may comprise one or more control subsystems, each control subsystem performing one or more functions assigned to the control system. The control subsystems may be provided entirely on one or more implantable parts of the system. Alternatively, the control subsystems may be provided entirely on one or more external parts of the system. Alternatively, the control subsystems may be distributed between one or more implantable parts of the system and one or more external parts of the system. The functions of the control system may be completely performed by one or more implantable components, the functions may be completely performed by one or more external components, or some of the functions may be performed by implantable components and some of the functions may be performed by external components. The control system may be configured to modify the signal produced by either wired or wireless communication with the signal generator.

In one aspect, a system for applying electrical stimulation to a nerve includes a implantable housing that includes a signal generator. The system further comprises an implantable neural interface for being in signaling contact with one or more nerves. The implantable neural interface further comprises at least a first electrode and a second electrode electrically coupled to the signal generator for applying a signal generated by the signal generator to the one or more nerves. The system further comprises an impedance measuring circuit electrically coupled to the first electrode and to the second electrode of the implantable neural interface and configured to measure an impedance of an electrical pathway therebetween. The system further comprises processing circuitry configured to periodically cause the impedance measuring circuit to measure impedances, the processing circuitry configured to determine a change in measured impedance that is indicative of an electrical event in the electrical pathway that includes one or both of the first electrode and the second electrode.

The housing may further include an electrically conductive portion for contacting tissue, in use, wherein the impedance measuring circuit is electrically coupled to the electrically conductive portion of the implantable housing to sense an impedance of an electrical pathway between the electrically conductive portion of the implantable housing and one or both of the first electrode and the second electrode.

In one aspect, a system for applying electrical stimulation to a nerve includes an implantable housing including a signal generator and one or more transducers for producing corresponding electrical fields based on the signals generated by the signal generator. The system comprises an implantable neural interface for being in signaling contact with one or more nerves, the implantable neural interface including one more transducers for producing a signal based on the electrical fields generated by the one or more transducers of the implantable housing. The system further comprises at least a first electrode and a second electrode electrically coupled to the one or more transducers for applying the signal produced by the one or more transducers to the one or more nerves. The implantable housing further includes an electrical field sensing circuit configured to sense a first electrical field produced by the first electrode and a second electrical field produced by the second electrode of the implantable neural interface. The systems further comprises processing circuitry configured to periodically cause the electrical field sensing circuit to sense electrical fields, the processing circuitry configured to determine a change in the sensed electrical fields that is indicative of an electrical event in one or both of the first electrode and the second electrode. The processing circuitry may be configured to detect a difference in sensed electrical field strength between a first electrical field comprising the first electrode and a second electrical field comprising the second electrode.

The signal generator may be configured to generate the signal in either a first mode, where the signal is applied between the first electrode and the second electrode, or in a second mode, where the signal is applied between the electrically conductive portion of the implantable housing and either the first electrode or the second electrode.

The control system may be operatively coupled to the signal generator and configured to cause the signal generator to switch from generating the signal in the first mode to generating the signal in the second mode in response to a determination by the processing circuitry that the change in measured impedance is indicative of an electrical event.

The control system may be is further configured to modify the signal generated by the generator in response to the determination by the processing circuitry that the change in measured impedance is indicative of an electrical event.

The system may also include a communication subsystem configured to couple to a remote device. The system may comprise a control system operatively coupled to the signal generator and configured to cause the communication subsystem to transmit an alert to the remote device in response to a determination by the processing circuitry that the change in sensed electrical fields is indicative of an electrical event.

The system may also include a control system operatively coupled to the signal generator and configured to modify the signal generated by the generator in response to a determination by the processing circuitry that the change in sensed electrical fields is indicative of an electrical event.

The or each transducer may be one or more of a coil, a Bluetooth transceiver, a nearfield communication transceiver and an ultrasound transducer.

The processing circuitry may be configured to generate data corresponding to said measured impedances or said sensed electrical fields over time and to determine the change in measured impedance from the generated data.

The electrical event may be one or more of a short circuit, and an electrical discontinuity.

The processing circuitry may be configured to calculate a baseline value of impedance from one or more of the measured impedances or said sensed electrical fields. Where the IPG is a part of a platform with internet connectivity as described above, data from more than one IPG may be analysed to determine a baseline value. The processing circuitry may be configured to determine a change in measured impedance by comparing the measured impedance with the baseline value of impedance. The change in measured impedance may comprise an increase and/or a decrease in impedance. The processing circuitry may be configured to determine if the magnitude of the change in measured impedance exceeds a first predetermined threshold of change in measured impedance. The processing circuitry may be further configured to determine if the magnitude of the change in measured impedance exceeds a second predetermined threshold of change in measured impedance. The first threshold may be greater than the second threshold. The processing circuitry may be configured to identify the presence of a first electrical event where the magnitude of the change in measured impedance exceeds the first predetermined threshold of change in measured impedance. The first electrical event may be one or more of a short circuit and an electrical discontinuity. The processing circuitry may be configured to identify the presence of a second electrical event where the change in measured impedance comprises a plurality of fluctuations and wherein the magnitude of each fluctuation exceeds the second predetermined threshold of change in measured impedance. The plurality of fluctuations may be irregular.
Comparing the magnitude of a change in impedance to two predetermined thresholds may be advantageous where intermittence indicative of an electrical fault is present in the measured impedances. In this case the magnitude of the change in impedance may be smaller than that of a significant electrical event, but greater than that of natural fluctuations that may exist in the system. A difference between the rolling average and the baseline impedance value may also be monitored and the z-score used to detect any abrupt change in the difference between the rolling average and the baseline impedance.

Using any one of or any different possible combination of the above mentioned detection of absolute change or relative changes, the performance of the implanted system may be monitored.

In one aspect, a method is provided. The method involves, in a system for applying electrical stimulation to a nerve, determining an electrical event in an electrical pathway that includes a first electrode of an implantable neural interface. The method includes periodically sensing an impedance in at least an electrical pathway between the first electrode and either a second electrode or an electrically conductive portion of a housing of the system, and determining an electrical event in the electrical pathway that includes the first electrode by identifying a change in the sensed impedance.

The step of determining an electrical event may include identifying a change in the rolling average impedance of the measured impedances, or the z-score.

The step of determining an electrical event may comprise calculating a baseline value of impedance from one or more of said sensed impedances. The step of determining an electrical event may comprise identifying a change in the sensed impedance by comparing the sensed impedance with the baseline value of impedance. The change in sensed impedance may comprise an increase and/or a decrease in impedance. The step of determining an electrical event may comprise determining if the magnitude of the change in sensed impedance exceeds a first predetermined threshold of change in sensed impedance. The step of determining an electrical event may further comprise determining if the magnitude of the change in sensed impedance exceeds a second predetermined threshold of change in sensed impedance. The first threshold may be greater than the second threshold. The presence of a first electrical event may be identified where the magnitude of the change in sensed impedance exceeds the first predetermined threshold. The first electrical event may be one or more of a short circuit and an electrical discontinuity. The presence of a second electrical event may be identified where the change in sensed impedance comprises a plurality of fluctuations and wherein the magnitude of each fluctuation exceeds the second predetermined threshold of change in sensed impedance. The plurality of fluctuations may be irregular.

The step of periodically sensing an impedance may further include periodically sensing a first impedance in a first electrical pathway between the electrically conductive portion of the implantable housing and the first array of electrodes and periodically sensing a second impedance in a second electrical pathway between the electrically conductive portion of the implantable housing and the second array of electrodes. The step of determining an electrical event in the first array of electrodes includes determining that the difference between the first impedance and the second impedance exceeds a predetermined threshold.

The step of periodically sensing an impedance may further include periodically sensing a first impedance in a first electrical pathway between the electrically conductive portion of the implantable housing and the second array of electrodes and periodically sensing a second impedance in a second electrical pathway between the first array of electrodes and the second array of electrodes. The step of determining an electrical event in the first array of electrodes may further include determining that the second impedance exceeds a predetermined threshold.

In another method, an electrical monitor such as an electrocardiogram (ECG) is used for intraoperative and/or post-operative monitoring. Stimulation artefact in the ECG signal that are caused by stimulation by an implantable device, for example one described above, may be identified and used to monitor the performance of the implantable device. Stimulation artefact in the signal from a lead placed closest to the neural interface may be analysed. The stimulation artefacts that concur with the stimulation signal from the implantable device in its starting time or duration may provide an indication that the implantable device is performing as expected. Identification of stimulation artefact in the ECG signal concurring with the stimulation signal from the implantable device may be used in combination with the impedance data analysis as described above.

In one aspect, a non-transitory computer-readable storage medium includes instructions that when executed by a computer, cause the computer to periodically sense an impedance in at least an electrical pathway between a first electrode and either a second electrode or an electrically conductive portion of a housing of a system, and determine an electrical event in the first electrode or the second electrode by identifying a change in the sensed impedance.

### BRIEF DESCRIPTION OF THE DRAWINGS

To easily identify the discussion of any particular element or act, the most significant digit or digits in a reference number refer to the figure number in which that element is first introduced.

FIG. 1A illustrates an implantable neural interface in the form of a neural cuff, suitable for use with a system according to the invention.

FIG. 1B illustrates the implantable neural interface of FIG. 1A.

FIG. 2 illustrates an implantable system in accordance with a first embodiment according to the invention.

FIG. 3 illustrates an implantable system in accordance with a second embodiment according to the invention.

FIG. 4 illustrates an implantable system in accordance with a third embodiment according to the invention.

FIG. 5 illustrates an implantable system in accordance with a fourth embodiment according to the invention.

FIG. 6A illustrates an exemplary embodiment of a graphical representation of time series data generated by a processing circuitry of an embodiment of the invention.

FIG. 6B illustrates first and second histograms of the time series of measured impedances of the first electrical pathway.

FIG. 6C illustrates a third histogram of the time series of measured impedances of the second electrical pathway.

FIG. 7A illustrates a graphical representation of data generated by a processing circuitry of an embodiment of the invention.

FIG. 7B illustrates a graphical representation of data generated by a processing circuitry of an embodiment of the invention.

### DETAILED DESCRIPTION

As used herein, an “array” of electrodes, such as the first array and the second array, refers to any two dimensional array of electrodes all of which are electrically coupled together at the same potential to stimulate a nerve or nerves. The electrodes of an array may be electrically connected in a linear array in series and/or parallel, though this need not be the case. For example, an array of electrodes may comprise four electrodes in a linear 1×4 array. However, an array of electrodes may comprise any two dimensional n by m array where n and m are positive integers and the electrodes of the array may be electrically connected in series and/or parallel. As described below, the present invention may also be applied to a neural interface with electrodes not in a form of arrays (i.e. a single electrode rather than an array of electrodes).

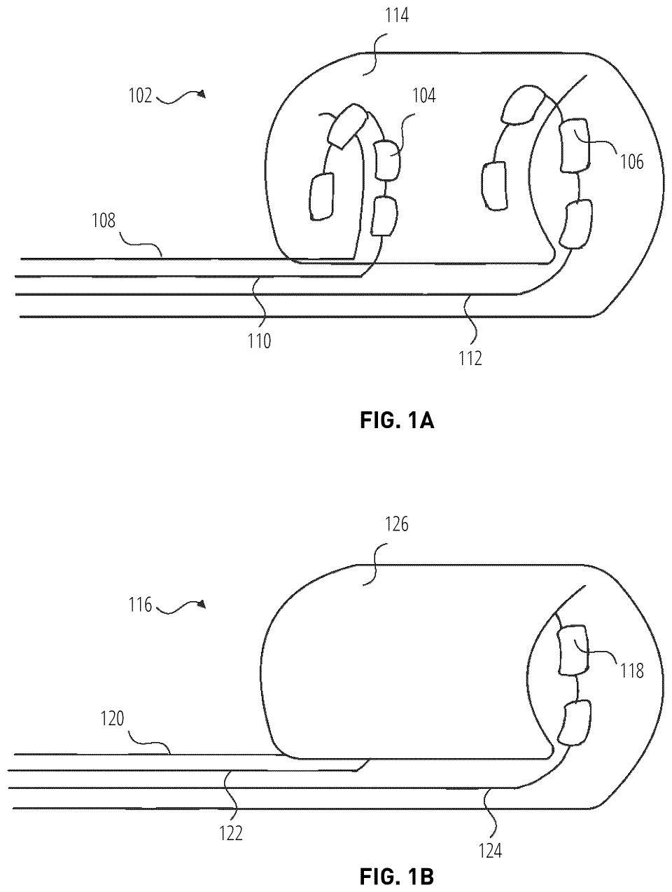

FIG. 1A shows an implantable neural interface 102 for use in a system of the present invention. The implantable neural interface 102 comprises a cuff comprising an arm 114, though more than one arm could be provided. The arm 114 is shown in cutaway such that the first array 104 and second array 106 of electrodes (described below) are visible. In particular, an embodiment of an implantable neural interface may be a cuff having two arms or a cuff having three arms. Other arms may be provided, and such arms may be active (i.e. comprising an electrode) or inactive (i.e. not comprising an electrode). Of course, the implantable neural interface need not be a cuff. Other implantable neural interfaces could be used, such as a continuous foil, and other implantable neural interfaces comprising a cuff could be used, such cuffs comprising a cylindrical structure.

The arm 114 of implantable neural interface 102 is configured to at least partially encircle a nerve (not shown). For the purposes of the present invention, the specific design of the implantable neural interface may take many different forms, such as a paddle electrode, spiral or helical electrode, cuff electrode, intravascular electrode, or stent electrode. In this example, the arm 114 of the implantable neural interface 102 comprises a first array 104 of electrodes, though it is not necessary to provide electrodes as an array, and individual electrodes, or one electrode or a continuous foil or other forms of electrode, could be provided instead. As shown, the first array 104 of electrodes comprises four electrodes, though more or fewer electrodes may be provided. The second array 106 of electrodes also comprises four electrodes, though again more or fewer electrodes may be provided, and again it is not necessary to provide electrodes as an array; individual electrodes, or even one electrode, could be provided instead. In this example, the first array 104 of electrodes is coupled to a first wire 110, which is coupled to a signal generator (not shown). The second array 106 of electrodes is coupled to a second wire 112, which again is coupled to a signal generator (not shown). The first wire 110 and the second wire 112 pass through a lead 108 to an implantable housing (not shown). Although two arrays of electrodes are shown in the implantable neural interface 102 of FIG. 1A, the invention does not require two arrays. To the contrary, an implantable neural interface for use in a system of the present invention may comprise just one array of electrodes, or it may comprise three or more arrays. In this particular embodiment, both arrays of electrodes are provided on its own arm, but again this is not essential and other arrangements are possible. Moreover, as described elsewhere herein, arrays may be provided in series or in parallel or in a combination of series and parallel.

FIG. 1B shows the identical implantable neural interface 116 to that shown in FIG. 1A, with an identical first array of electrodes (not shown), second array 118 of electrodes, lead 120, first wire 122, second wire 124 and arm 126 as shown in FIG. 1A. The only difference between FIG. 1A and FIG. 1B is that arm 126 is shown in solid.

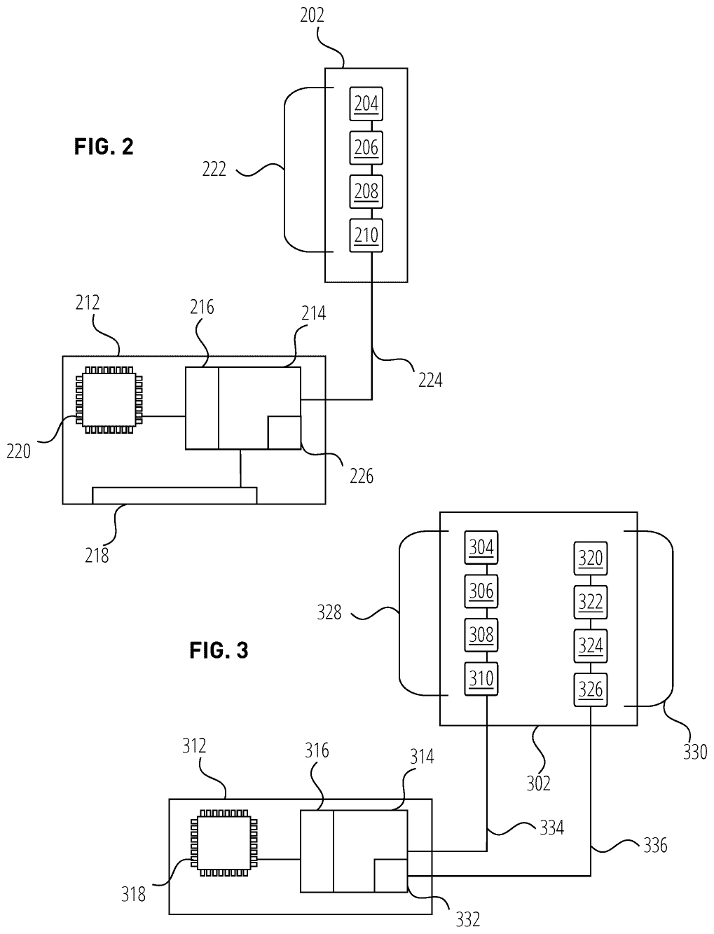

FIG. 2 shows an embodiment of a system according to the present invention for applying electrical stimulation to a nerve (not shown). The system comprises an implantable neural interface 202, which may be similar to the implantable neural interface 102 design of FIG. 1A except that in the embodiment of FIG. 2 the implantable neural interface 202 comprises only a first array 222 of electrodes, and not a second array. The first array 222 of electrodes comprises a first electrode 204, a second electrode 206, a third electrode 208, and a fourth electrode 210, though more or fewer electrodes (even a single electrode) may be provided. Although the first array 222 is shown with four electrodes connected in series, it is possible to provide electrodes that are connected in parallel. It will be appreciated that if an electrical event such as a break or shorting occurs, and where these electrodes are connected in series, the functionality or performance of the array will depend upon where the break has occurred. If the connection in the electrical connector between the electrode array and the signal generator (also referred to as the lead 224) has an electrical event such as a break, the whole array may not function. However, if the electrical event is in electrode 204 (furthest from the signal generator) or between electrode 204 and 206, then first three electrodes will still be functional. If these electrodes were to be connected in parallel, it will be appreciated that a break in an electrode or in electrical connections between electrodes will leave the remaining electrodes functional.

The system comprises an implantable housing 212 coupled to the implantable neural interface 202 by a lead 224. The lead 224 may be of any length, and may only comprise of the electrical conductors in the neural interface 202 and the implantable housing 212. In other embodiments, where the neural interface 202 and the implantable housing 212 are in separated locations, the lead 224 may be longer with portions external to the neural interface 202 and the implantable housing 212. The implantable housing 212 comprises a signal generator 214, an impedance sensing circuit 226, a control system 216 and processing circuitry 220, each of which is described in more detail below.

The implantable housing 212 further comprises an electrically conductive portion 218 which is configured, in use, to contact surrounding tissue (not shown). The electrically conductive portion 218 may be an electrode fixed to the outer surface of the implantable housing 212, or it may be formed by some or all of the structure of the implantable housing 212 itself. The electrically conductive portion 218 is configured such that it is electrically coupled to the signal generator 214 to act as a return electrode for the first array 222 of electrodes. The configuration shown in FIG. 2 comprising a first array 222 of electrodes and an electrically conductive portion 218 of the implantable housing 212 is an example of a monopolar configuration of a system according to the present invention.

The signal generator 214 is configured to generate one or more electrical signals for application to a nerve (not shown) via the first array 222 of electrodes on the implantable neural interface 202. The nature of the electrical signals is not germane to the present invention and is not discussed herein except to state that it can take any suitable form known in the art.

The impedance sensing circuit 226 may take any conventional form known to those skilled in the art, for example a current source (not shown) and a voltmeter (not shown) such that impedance can be measured using the equation R=V/I The impedance sensing circuit 226 is configured to sense impedance through an electrical pathway comprising the signal generator 214, the wire (not shown) passing through the lead 224; the first array 222 of electrodes, including the first electrode 204, second electrode 206, third electrode 208 and fourth electrode 210; the nerve or nerves (not shown) to which the signal is applied, and the tissue surrounding the nerve or nerves and the implantable housing 212; and finally the electrically conductive portion 218 of the implantable housing 212 from which the signal returns back to the signal generator 214.

The control system 216 is electrically coupled to the signal generator 214 and is capable of modifying the signal generated by the signal generator 214. A signal may be modified in different ways, including by modifying its amplitude, frequency and/or phase; and in the case of a pulsed signal, by modifying its amplitude, frequency, phase, duty cycle and/or pulse width. The control system 216 is also capable of selecting which electrode or electrode array is provided with the original or modified signal, or of deactivating the signal generator 214.

The processing circuitry 220 is coupled to the impedance sensing circuit 226, the control system 216 and the signal generator 214, and may be configured to perform several functions. Firstly, the processing circuitry 220 may cause the signal generator 214 to apply the electrical signal to the first array 222 of electrodes. Secondly, the processing circuitry 220 may cause the impedance sensing circuit 226 to sense the impedance through the electrical pathway as described above. The processing circuitry 220 may cause the impedance sensing circuit 226 to sense the impedance of the electrical pathway on an ad-hoc basis or periodically. Alternatively, the processing circuitry 220 may cause the impedance sensing circuit 226 to sense the impedance once, on an ad-hoc basis, periodically or continuously, and impedance may be calculated on an ad-hoc basis or periodically. It is to be appreciated that reference to the sensing or measuring of impedance may also encompass the subsequent calculation of the impedance. The processing circuitry may cause the impedance sensing circuit to sense the impedance once a day, however, greater or lesser frequencies of sensing, for example twice a day, once every two days, once a week etc., are possible. The processing circuitry may cause the impedance sensing circuit to sense the impedance at a certain time (or times) of day. In some cases, the time may be chosen such that the impedance sensing coincides with when the patient is sleeping or resting. In other cases, impedance data may be collected before and after each stimulation. Any combination of impedance data collection trigger may be used, for example the impedance data may be collected before and after each stimulation and in addition at particular times of the day. The processing circuitry may cause the impedance sensing circuit to sense the impedance at the same time (or times) each day. By way of example, the impedance may be sensed at a certain time overnight, when it is expected the patient is sleeping. It is also possible for the processing circuitry to apply weightings to the impedance measurements. Certain impedance measurements may be considered more significant than others and accordingly these measurements may be given a higher weighting such that they have more significance in any subsequent data processing or analysis. For example, in a series of measurements taken each hour over a 24-hour period, those coinciding with when the patient is substantially stationary may be given a higher weighting and/or considered more significance in subsequent data processing and analysis, than those taken when the patient is active. The implantable system may also comprise an accelerometer such that it is possible to obtain both impedance data and acceleration data. Similarly, the impedance data may be considered in combination with data from a wearable device to obtain more information about the patient's status when the impedance data was measured. As another example, the data measured whilst the patient was relatively still (for example determined based on acceleration data or other activity data) may be given higher weighting over the data that was measured whilst the patient was relatively more active. As another example, the data from the accelerometer (or other sensors in the IPG or the wearable device) may be used to determine a postural state of the patient. The impedance data measured whilst the patient is in a known postural state considered more stable may be provided a higher weighting. Instead of (or in addition to) providing different weighting, impedance data measurement may occur during a certain time, activity or posture. The processing circuitry 220 may be configured to generate data corresponding to the measured impedance. For example, the processing circuitry 220 may cause the impedance sensing circuit 226 to sense impedance of the electrical pathway once per hour, and generate a time series of the sensed impedance showing changes to the measured impedance over time. The processing circuitry may be configured to generate a time series of the sensed impedance for any frequency of measured impedance. A time series of sensed impedance where each data point represents the impedance measured at the same time each day to coordinate with a time where the patient is substantially stationary, for example sleeping. In a time series of impedance measurements taken at a frequency of greater than once a day, a higher weighting may be applied to those measurements coinciding with when the patient is substantially stationary. The processing circuitry may be configured to increase the frequency of the impedance measurement once a potential electrical event is detected. The processing circuitry 220 may be coupled to a memory (not shown) in which to store the generated data. Thirdly, the processing circuitry 220 may cause the control system 216 to control the signal generator 214 based on the generated data, as explained in more detail below. Depending on the measured impedance, the frequency of the impedance sensing may also be modified by the control system 216 or the processing circuitry 220.

FIG. 3 shows an embodiment of a system according to the present invention for applying electrical stimulation to a nerve (not shown). In many ways, the system of FIG. 3 is similar to the system of FIG. 2, in that it comprises an implantable neural interface 302, an implantable housing 312, a signal generator 314, a control system 316, processing circuitry 318, and an impedance sensing circuit 332. To the extent that these features may be configured in the same manner as the corresponding features in the embodiment of FIG. 2, they will not be described again.

The first array 328 of electrodes comprises a first electrode 304, a second electrode 306, a third electrode 308, and a fourth electrode 310. In addition to the first array 328, the implantable neural interface 302 of the embodiment of FIG. 3 comprises a second array 330 of electrodes comprising a first electrode 320, a second electrode 322, a third electrode 324 and a fourth electrode 326. The first array 328 of electrodes and the second array 330 of electrodes may be configured in the same way as the corresponding features of FIG. 1A. The second array 330 of electrodes is similar to the first array 328 of electrodes, insofar as it is a linear array of four electrodes, though this need not be the case and the second array 330 of electrodes may have a different configuration from the first array 328 of electrodes. As with the first array 328 of electrodes, the second array 330 of electrodes is also electrically coupled to the signal generator 314.

Unlike the embodiment of FIG. 2, the implantable housing 312 does not comprise an electrically conductive portion that acts as a return electrode. Instead one of the first array 328 and the second array 330 of electrodes acts as the return. The configuration shown in FIG. 3 comprising a first array 328 of electrodes and second array 330 of electrodes is an example of a bipolar configuration of a system according to the present invention.

In view of the different configuration of electrodes, the impedance sensing circuit 332 of FIG. 3 is configured to sense impedance through a different electrical pathway compared with that of FIG. 2. In particular, the electrical pathway of FIG. 3 comprises the signal generator 314, the wire (not shown) passing through the first lead 334; the first array 328 of electrodes, including the first electrode 304, second electrode 306, third electrode 308 and fourth electrode 310; the nerve or nerves (not shown) to which the signal is applied; the second array 330 of electrodes, including the first electrode 320, second electrode 322, third electrode 324 and fourth electrode 326; and finally the second lead 336 from which the signal returns back to the signal generator 314.

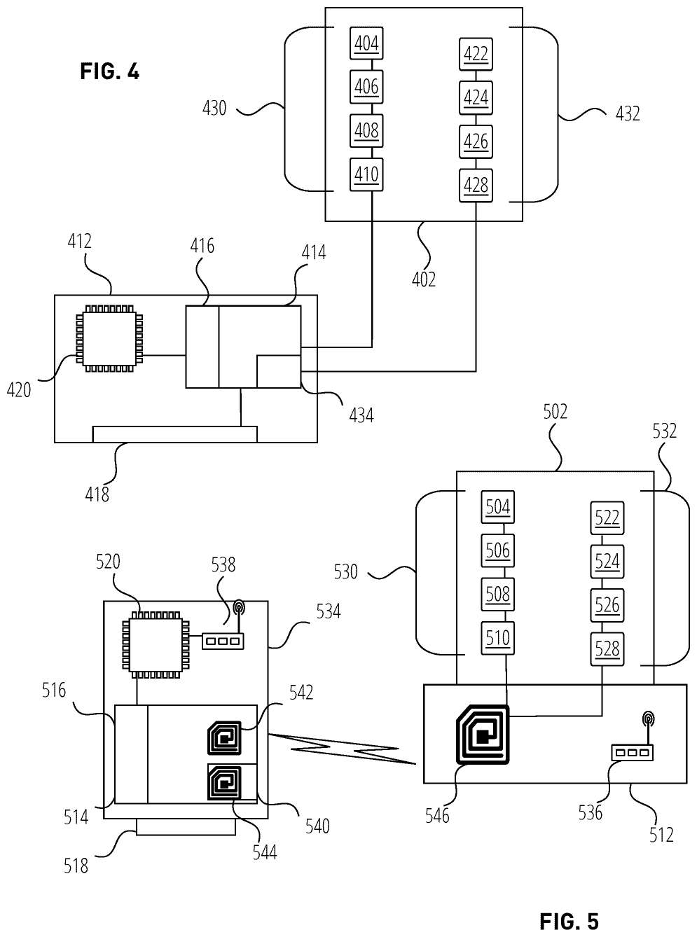

FIG. 4 shows an embodiment of a system according to the present invention for applying electrical stimulation to a nerve (not shown). In many ways, the system of FIG. 4 is similar to the system of FIG. 2 and the system of FIG. 3, in that it comprises an implantable neural interface 402, an implantable housing 412, a signal generator 414, a control system 416, an electrically conductive portion 418, processing circuitry 420, an impedance sensing circuit 434, a first array 430 of electrodes and a second array 432 of electrodes. To the extent that these features are configured in the same manner as the corresponding features in the embodiment of FIG. 2 and FIG. 3, they will not be described again.

It will be apparent that the embodiment of FIG. 4 comprises a first array 430 and a second array 432 of electrodes in a similar manner to the embodiment of FIG. 3. In particular, the first array 430 of electrodes comprises a first electrode 404, a second electrode 406, a third electrode 408, and a fourth electrode 410. The second array 432 of electrodes comprises a first electrode 422, a second electrode 424, a third electrode 426 and a fourth electrode 428. The first array 430 of electrodes and the second array 432 of electrodes may be configured in the same way as the corresponding features of FIG. 1A. The second array 432 of electrodes is similar to the first array 430 of electrodes, insofar as it is a linear array of four electrodes, though this need not be the case and the second array 432 of electrodes may have a different configuration from the first array 430 of electrodes. As with the first array 430 of electrodes, the second array 432 of electrodes is also electrically coupled to the signal generator 414.

It will also be appreciated that the embodiment of FIG. 4 comprises an electrically conductive portion 418 in a similar manner to the embodiment of FIG. 2.

In view of the different configuration of electrodes, the impedance sensing circuit 434 of FIG. 4 is configured to sense impedance through a different electrical pathway compared to the other embodiments. In fact, what follows from the configuration described above is that the embodiment of FIG. 4 does not have just one electrical pathway but three.

The first electrical pathway is the same as the electrical pathway of the embodiment of FIG. 3, and runs in a bipolar configuration from the signal generator 414 to the first array 430 of electrodes, then the second array 432 of electrodes and back to the signal generator 414. The second electrical pathway and the third electrical pathway are similar to the electrical pathway of the embodiment of FIG. 2, and run in a monopolar configuration from the signal generator 414 to the first array 430 of electrodes or the second array 432 of electrodes, respectively, then to the electrically conductive portion 418 and back to the signal generator 414. It will be appreciated that the second and third electrical pathways can be separate (i.e the signal can pass between the first array 430 of electrodes and the electrically conductive portion 418 but not the second array 432 of electrodes or vice versa) or they can be the same (i.e the signal can pass between the first array 430 and the second array 432 of electrodes, and the electrically conductive portion 418).

FIG. 5 shows an embodiment of a system according to the present invention for applying electrical stimulation to a nerve (not shown). In many ways, the system of FIG. 5 is similar to the system of FIG. 4, in that it comprises an implantable neural interface 502, an implantable housing 512, a signal generator 514, a control system 516, an electrically conductive portion 518, processing circuitry 520, an impedance sensing circuit 540, a first array 530 of electrodes and a second array 532 of electrodes. To the extent that these features are configured in the same manner as the corresponding features in the embodiment of FIG. 4, they will not be described again.

The embodiment of FIG. 5 differs from that of FIG. 4 insofar as the implantable housing 512 and the implantable neural interface 502 are provided as a single integrated circuit or system on a chip that is unconnected from the signal generator 514. In this case, the implantable housing 512 does not comprise signal generator 514, control system 516, electrically conductive portion 518 or processing circuitry 520, which are instead provided on a remote device 534. Remote device 534 may be implanted in the patient at a location that is in signaling communication with the implantable housing 512, such as subcutaneously or at any convenient location near to implantable housing 512. Alternatively, remote device 534 may be positioned at a location external to the patient but still in signaling communication with the implantable housing 512, in which case it may not include electrically conductive portion 518.

Remote device 534 comprises processing circuitry 520 coupled to control system 516 and to a receiver 538 which is communicatively coupled to a transmitter 536 in the implantable housing 512. In some embodiments of wireless configurations, of which FIG. 5 is one example, implantable housing 512 may comprise an impedance sensing circuit (not shown) that is configured to sense impedance in the manner described above. In such cases, transmitter 536 can transmit information from the impedance sensing circuit (not shown) located in the implantable housing 512 and transmit it to receiver 538 which communicates it to processing circuitry 520. Of course, either or both of receiver 538 and transmitter 536 can be transceivers and configured to send and receive information from implantable housing 512 to remote device 534 and from remote device 534 to implantable housing 512.

In the example of FIG. 5, signal generator 514 comprises a coil 542 (though more than one coil may be provided), and implantable housing 512 comprises a coil 546 (though, again, more than one coil may be provided). Coil 546 is electrically coupled to first array 530 of electrodes and second array 532 of electrodes, though where implantable housing 512 comprises multiple coils, each array of electrodes may be connected to its own coil. Coil 542 transmits power wirelessly to coil 546, which causes the signal generated at signal generator 514 to pass to the nerve via the implantable neural interface 502 in the same manner described above, albeit having been transmitted wirelessly from signal generator 514 via coil 542 and coil 546 to implantable housing 512. In other embodiments, other powering modalities may be used, such as Bluetooth, near field communication, ultrasound and so on. For example, the neural interface 502 may comprise a transducer for receiving energy from an external source.

Impedance sensing circuit 540 also may comprise coil 544 (though more than one coil may be provided), which can be used to determine impedance by sensing electrical fields generated in implantable housing 512. Once impedance is determined in this way, the configuration of FIG. 5 operates in the same manner as previous embodiments.

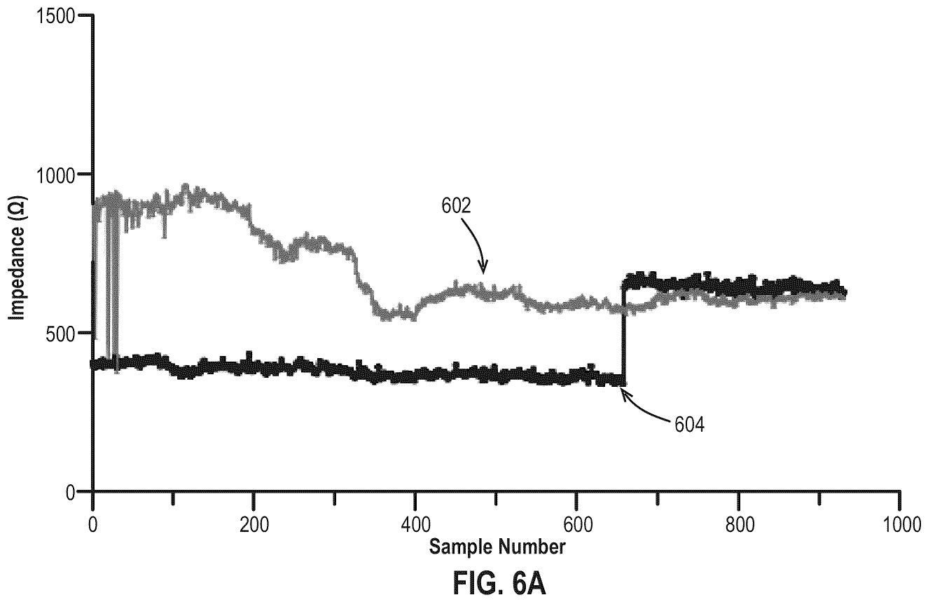

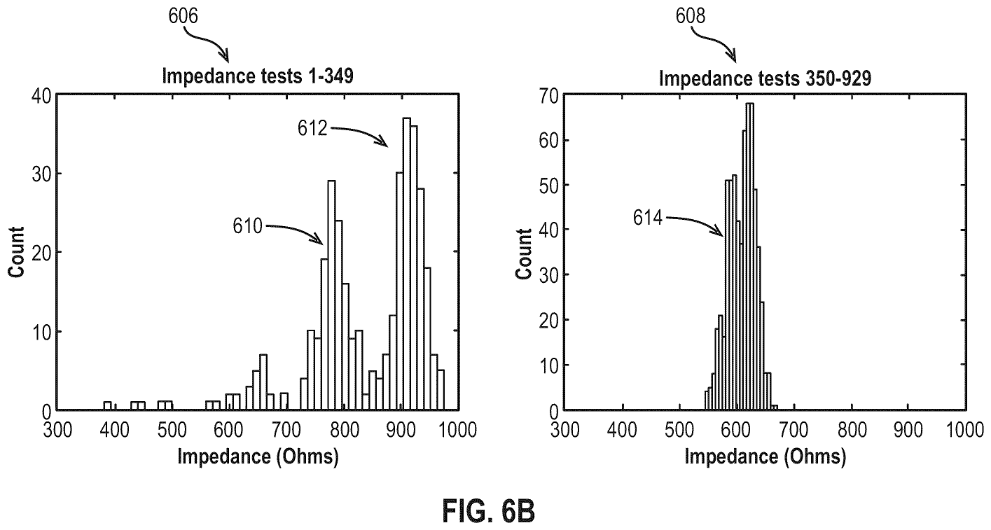

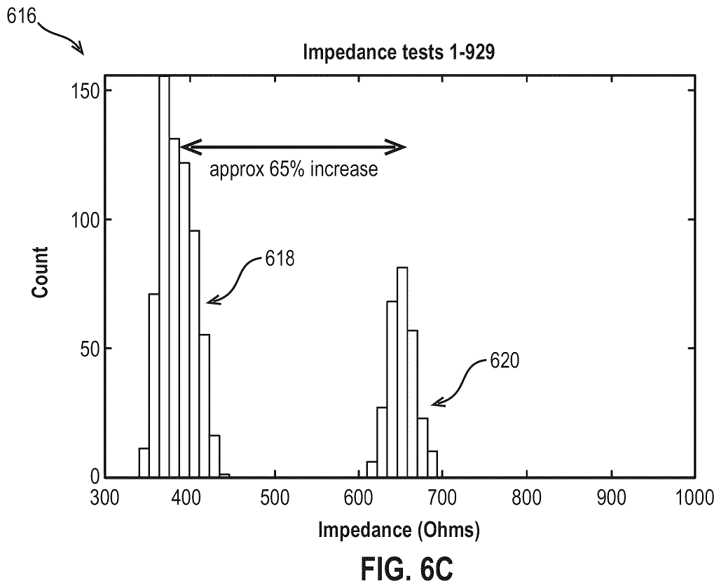

FIG. 6A is an exemplary embodiment of a graphical representation of time series data generated by a processing circuitry of any of the embodiments of FIG. 3 to FIG. 5. The graph shows changes to the measured impedance over time of two electrical pathways. Trace 602 shows changes to the measured impedance over time of a first electrical pathway that includes a first array of electrodes and an electrically conductive portion of the implantable housing, as described above in connection with any of the embodiments of FIG. 3 to FIG. 5. Trace 604 shows changes to the measured impedance over time of a second electrical pathway that includes a second array of electrodes and an electrically conductive portion of the implantable housing, as described above in connection with any of the embodiments of FIG. 3 to FIG. 5. For example, the first array may be a proximal array and the second array may be a distal array, or vice versa.

The configuration described above is an example of a monopolar configuration where each of the first electrical pathway and the second electrical pathway represents a separate monopolar pathway involving one of the first array of electrodes and the second array of electrodes respectively, and the electrically conductive portion of the implantable housing. Measured impedance can be determined using a rolling average, or other suitable statistical data analysis technique. The processing circuitry may be configured to calculate a baseline value of impedance from one or more of the measured impedance values or the sensed electrical fields. The baseline value of impedance may be the average of the impedance values taken during the first few days after implantation of the system. However, other time frames over which the impedance values may be averaged are possible. The first impedance measurement of the series of measurements included in the calculation of the baseline value may be the first impedance measurement taken after implantation. Alternatively, it may be a measurement taken a few days after implantation of the system. An exemple baseline impedance value for a system implanted may be 400-600 Ohms. This impedance value is purely exemplary and other baseline impedance values are possible. A singular baseline value of impedance may be calculated for the system, or a baseline value of impedance may be determined individually for one or more of each electrical pathway. The processing circuitry may be configured to determine a change in measured impedance by comparing the measured impedance with the baseline value of impedance. Such a change may indicate the presence of an electrical event. The measured impedance may increase or decrease relative to the baseline value. The measured impedance may also fluctuate relative to the baseline value, as is discussed in more detail later. Such fluctuations involve both increases and decreases in measured impedance relative to the baseline value. By way of example, the relative increase or decrease in impedance from the baseline value may be a change in impedance of 20%, 25%. 30%, 35%, 40%, 45%, 50%, 55%, 60%, 65%, 70%, however greater or lesser percentage changes are possible. A change using rolling average of the impedance data and z-score measured in terms of deviation from the average may also be used to determine a potential electrical event. The z-score may be used to detect any abrupt change in the rolling average. The z-score may act as a threshold to trigger detection of an electrical event.

The graph plots the measured impedance for each of the first and second electrical pathways for each of 929 measurements. In other words, the data generated by the processing circuitry that lead to this time series follows from 929 separate sample where the impedance measuring circuit has sensed the impedance of the first and second electrical pathways. By way of example, if the processing circuitry is configured to cause the impedance sensing circuit to sense impedance once per hour (i.e. 24 times per day), then the data shown in the graph represents sensing over a period of more than 38 days. Of course, different time periods are possible; for example, 12 times per day; 48 times per day or continuously such as every second or every minute. Less frequent time periods are also possible, such as once a day, once every two days, once a week or once a month.

It will be appreciated from studying trace 602 that the measured impedance of the first electrical pathway changes at around sample number 350. In particular it can be seen that having fluctuated for approximately 200 samples at around 900 ohms and for a further 150 samples at around 800 ohms, the impedance drops to around 600 ohms where it is maintained for the rest of the period. Likewise it will be appreciated from studying trace 604 that the measured impedance of the second electrical pathway changes significantly at around sample number 650. In particular it can be seen that having fluctuated for approximately 650 samples at around 400 ohms, the impedance suddenly rises significantly to around 650 ohms.

FIG. 6B shows first histogram 606 of the time series of measured impedances of the first electrical pathway from samples 1 to 349 and a second histogram 608 of the time series of measured impedances of the first electrical pathway from samples 350 to 929. The first histogram 606 makes apparent the change in impedance from around 900 ohms to around 800 ohms. Comparing the first histogram 606 with the second histogram 608, the change in impedance from around 800 ohms to around 600 ohms is also made apparent. Each of the changes in impedance is demarcated by a peak of the histograms. In particular, a first peak 610 shows the impedance in the first electrical pathway from approximately sample 200 to approximately sample 350. A second peak 612 shows the impedance in the first electrical pathway from sample 1 to approximately sample 200. Finally, the third peak 614 shows the impedance in the first electrical pathway from sample 350 to sample 929.

Each change in impedance represents a possible emergence of an electrical event, with the magnitude of the change being indicative of the likelihood and/or the extent of the electrical event. For example, the reduction in impedance from around 900 ohms (i.e the first peak 610) to around 800 ohms (i.e the second peak 612) represents a reduction of just over 10%. By contrast, the reduction in impedance to around 600 ohms (i.e the third peak 614) represents a reduction of 25% from the second peak 612 and a reduction of 33% from the first peak 610. The change need not be an increase in impedance and a decrease could also indicate a possible emergence of an electrical event, although an increase is more likely to be an indication of a possible emergence of an electrical event such as an electrical discontinuity (such as a break) or a short circuit, or an indication of a more significant electrical defect. Further, the amount of change in the impedance may be an indication of the extent of the electrical defect. For example, the impedance change may be higher when more electrodes are lost due to the location or number of the electrical defect(s).

It will be appreciated that processing circuitry may be configured to determine the emergence of an electrical event based on the magnitude in the change in impedance compared with a predetermined threshold. The predetermined threshold may be a change in impedance measured relative to the baseline value of impedance. For instance, if a predetermined threshold is set at 15%, then an emergence of an electrical event will not be determined during samples 1 to 349, but will be determined on or around sample 350 when the impedance drops by a significant amount (i.e by an amount in excess of the predetermined threshold). The predetermined threshold of 15% may be comprise a 15% increase or a 15% decrease in impedance from the baseline value. The processing circuitry may be configured to determine if the magnitude of the change in measured impedance exceeds one or both of first and second predetermined thresholds of change in measured impedance. However, having more than two predetermined thresholds is possible. The magnitude of the change in impedance may be indicative of the likelihood and/or the extent of the electrical event, and accordingly the predetermined threshold values may be set to identify electrical faults of varying extent or significance. A higher predetermined threshold may be used to identify a more significant or extensive electrical event than a lower predetermined threshold. The processing circuitry may be configured to determine if the magnitude of the change in measured impedance exceeds one or both of first and second predetermined thresholds of change in measured impedance. The two thresholds are set at differing magnitudes of change in impedance, for example the first threshold may be greater than the second threshold. The processing circuitry may be configured to identify the presence of a first electrical event where the magnitude of the change in impedance exceeds the first predetermined threshold of change in measured impedance. The change in impedance may be reflected by a step change in impedance, for example a step increase or step decrease. It is also possible for changes in impedance with a magnitude lower than a threshold value indicative of an electrical defect in the system. These changes can be characterised by intermittent fluctuations which may comprise an irregular or regular pattern of any number of increases and decreases in impedance. Intermittency may be distinguished from natural fluctuations in the system, which may have a more regular pattern over a longer period of time. The magnitude of the change in impedance caused by natural fluctuations may also be smaller in magnitude than fluctuations indicative of an electrical fault. A second predetermined threshold which is smaller in magnitude than the first predetermined threshold may enable the processing circuitry to determine the presence of intermittency indicative of an electrical event. The processing circuitry may be configured to identify a change in measured impedance comprises a plurality of fluctuations and wherein the magnitude of each fluctuation exceeds the second predetermined threshold of change in measured impedance. The change may be characterised by a plurality of fluctuations in measured impedance. During a fluctuation, the measured impedance values may be within the predetermined absolute threshold value or predetermined percentage threshold in a relative change to a baseline value. The fluctuation may be detected when there is a series of rising and falling impedance values over a predetermined period of time. A fluctuation determining threshold values may be provided to detect such fluctuation. The fluctuation determining threshold value or percentage threshold will generally be within, like a subset of, the predetermined threshold value or percentage range for determining an electrical event. For example, a predetermined absolute threshold value may be 1000 ohms, and a fluctuation determining absolute threshold values may be 200 ohms and 500 ohms, wherein impedance values falling below 200 ohms and those rising above 500 ohms may be detected. As another example, a predetermined relative percentage change threshold for determining an electrical event may be 60%, and a fluctuation determining relative percentage change threshold may be 20% and 40%, wherein impedance values that change less than 20% and more than 40% are detected. It may also be possible to include a repeatability assessment over time of an event occurring, to reduce detection of false positives on electrical events which may occur if based only on a single fluctuation. For example, a repetitive measure of the event may be added, with reliability within a set variance of percentage change or value.

FIG. 6C shows a third histogram 616 of the time series of measured impedances of the second electrical pathway from samples 1 to 929. The third histogram 616 makes apparent the change in impedance from around 400 ohms to around 650 ohms. As before, the change in impedance is demarcated by a peak of the histogram. In particular, a fourth peak 618 shows the impedance in the second electrical pathway from sample 1 to approximately sample 650. A fifth peak 620 shows the impedance in the second electrical pathway from approximate sample 650 to sample 929.

As before, the change in impedance represents the likely emergence of an electrical event, with the magnitude of the change being indicative of the likelihood of the electrical event. In this case, the increase in impedance from around 400 ohms (i.e the fourth peak 618) to around 650 ohms (i.e the fifth peak 620) represents an increase of around 65%.

As before, it will be appreciated that processing circuitry may be configured to determine the emergence of an electrical event based on the magnitude in the change in impedance compared with a predetermined threshold. For instance, if a predetermined threshold is set at 15%, then an emergence of an electrical event will be determined on or around sample 650 when the impedance increases by a significant amount (i.e by an amount in excess of the predetermined threshold).

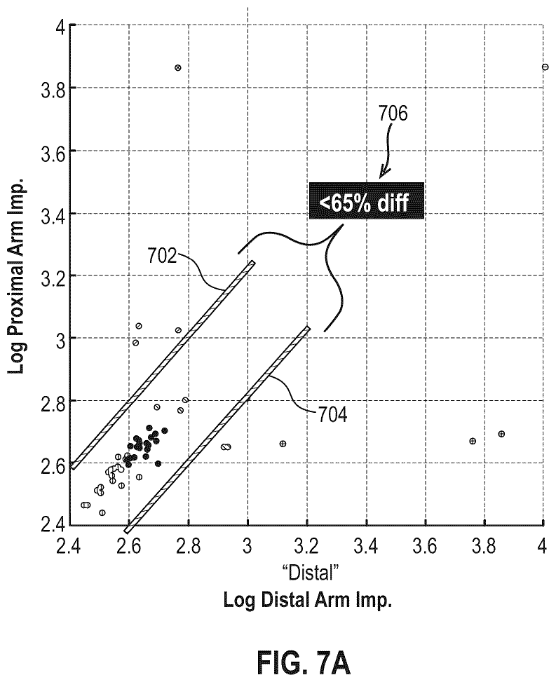

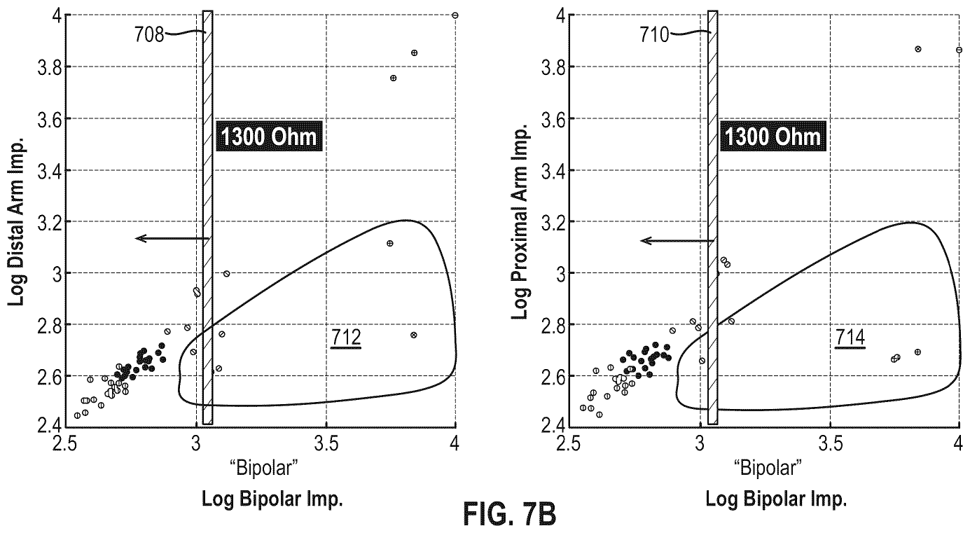

FIG. 7A is another exemplary embodiment of a graphical representation of data generated by a processing circuitry of any of the embodiments of FIG. 3 to FIG. 5. Each point on the graph shows the measured impedance of two electrical pathways at a point in time. The y-axis shows the measured impedance of a first electrical pathway that includes a first array of electrodes and an electrically conductive portion of the implantable housing, as described above in connection with any of the embodiments of FIG. 3 to FIG. 5. The x-axis shows the measured impedance of a second electrical pathway that includes a second array of electrodes and an electrically conductive portion of the implantable housing, as described above in connection with any of the embodiments of FIG. 3 to FIG. 5. As shown, the first array is a proximal array and the second array is a distal array.

The configuration described above is another example of a monopolar configuration where each of the first electrical pathway and the second electrical pathway represents a separate monopolar pathway involving one of the first array of electrodes and the second array of electrodes respectively, and the electrically conductive portion of the implantable housing.

The graph plots the measured impedance for each of the first and second electrical pathways, with each point representing a point in time. Where the impedance in the first electrical pathway is approximately the same as the impedance in the second electrical pathway, the point will lie on the line X=Y. The greater the difference between the impedances of the first and second electrical pathways, the further away the point will be from the line X=Y. If there are no detected electrical events in either the first array of electrodes or the second array of electrodes, the impedance of the first and second electrical pathways will be approximately equal. On the other hand, if one or other of the first and second arrays of electrodes has a detectable electrical event, the impedance of the first and second electrical pathways will differ. The greater the number of electrical events in one or other of the first and second electrical pathways, the greater the difference will be. To put it another way, below a certain threshold, if there is no electrical event in either the first array or electrodes, or the second array of electrodes, the impedance of both the arms should be approximately equal.

FIG. 7A shows a first threshold 702 and a second threshold 704 which graphically illustrate the maximum permitted difference between the electrical impedances—in this case a difference 706 of no more that 65%—beyond which an electrical event will be determined. In other words, where a point on the graph falls between the first threshold 702 and the second threshold 704, the difference in impedance between the first and second electrical pathways will be less than the predetermined threshold of difference 706, and no electrical event will be determined. Where a point on the graph falls above first threshold 702, the impedance of the first array will be significantly higher than the impedance of the second array, indicating an electrical event in the first array. Where a point on the graph falls below second threshold 704, the impedance of the second array of electrodes will be significantly higher than the impedance of the first array, indicating an electrical event in the second array.

FIG. 7B is another exemplary embodiment of graphical representations of data generated by a processing circuitry of either of the embodiments of FIG. 4 or FIG. 5. As with FIG. 7A, each point on the graphs show the measured impedance of two electrical pathways at a point in time. The x-axis of each graph shows the measured impedance of a first electrical pathway that includes a first array of electrodes and second array of electrodes, as described above in connection with either of the embodiments of FIG. 4 to FIG. 5. The y-axis of the left hand graph shows the measured impedance of a second electrical pathway that includes a second array of electrodes and an electrically conductive portion of the implantable housing, as described above in connection with either of the embodiments of FIG. 4 to FIG. 5. The y-axis of the right hand graph shows the measured impedance of a second electrical pathway that includes a first array of electrodes and an electrically conductive portion of the implantable housing, as described above in connection with either of the embodiments of FIG. 4 to FIG. 5. As shown, the first array is a proximal array and the second array is a distal array.

The configuration described above is an example of a bipolar configuration where the second electrical pathway represents a bipolar pathway comprising both the first array of electrodes and the second array of electrodes. As will be appreciated, the first electrical pathway represents a monopolar pathway involving one of the first array of electrodes and the second array of electrodes respectively, and the electrically conductive portion of the implantable housing.

The graph plots the measured impedance for each of the first and second electrical pathways, with each point representing a point in time. Where the impedance in the first electrical pathway is greater than a threshold impedance, it can be determined that an electrical event has emerged, since the impedance of an electrical pathway that involves the first array of electrodes and the second array of electrodes should not exceed a certain threshold amount. Where an electrical event has been determined based on the impedance in the first electrical pathway exceeding a predetermined threshold but the impedance of the second electrical pathway remains at normal levels (for example, beneath the line X=Y), then it can be determined that the electrical event exists in the array of electrodes that is not in the second electrical pathway.

FIG. 7B shows a bipolar impedance threshold 708 and 710, to the right of which indicates a likely emergence of an electrical event. The left hand graph shows a first threshold region 712 to the right of the bipolar impedance threshold 708 and the right hand graph shows a second threshold region 714 to the right of the bipolar impedance threshold 710. Each of the first threshold region 712 and the second threshold region 714 lies below the line X=Y, indicating a normal measured impedance for the second electrical pathway. Accordingly, where the second electrical pathway comprises the second array of electrodes (left hand graph), a point in the first threshold region 712 indicates an electrical event in the first array of electrodes. Where the second electrical pathway comprises the first array of electrodes (right hand graph), a point in the second threshold region 714 indicates an electrical event in the second array of electrodes. As shown, the first array is a proximal array and the second array is a distal array.

In embodiments of the invention not shown. The implantable housing and the implantable neural interface need not be connected by a lead. Instead, the implantable neural interface may provide its own power and be controlled by the implantable housing. In such cases, the invention may be implemented by sensing electrical fields in order to determine impedance in the electrical pathways.

As described above in connection with the embodiments of FIG. 2 to FIG. 5, the processing circuitry is configured to cause the control system to modify the signal generated by the signal generator based on the generated data. For example, if an electrical event is determined, the processing circuitry may cause the control system to: modify the signal (by modifying one or more of the amplitude, frequency and/or phase; and in the case of a pulsed signal, by modifying its amplitude, frequency, phase, duty cycle and/or pulse width) based on the data generated (for example, the signal may be reduced by a predetermined amount, or by an amount that reflects the significance of the electrical defect indicative of the data generated); send signal to only a selective set of electrode(s) or electrode array(s) based on the data generated; or deactivate the signal generator.

As described above in connection with the embodiments of FIG. 4 or FIG. 5, the processing circuitry may be configured to cause the control system to change the electrical pathway used by the signal generator. For example, if the processing circuitry determines the emergence of an electrical event in one of the first array of electrodes or the second array of electrodes, it may cease to operate in a bipolar configuration by sending a signal between the first and second arrays of electrodes and instead operate in a monopolar configuration by sending a signal between one of the first and second arrays of electrodes (i.e, the array that does not have an electrical event) and the electrically conductive portion of the implantable housing. In embodiments where there are more than two arrays, the system may continue to operate in bipolar configuration even if the signal is no longer sent to one of the arrays. In embodiments where the electrodes or electrode arrays are connected in parallel, the signal may be sent to electrodes or electrode arrays that are determined to be in operation and thus continue to operate in bipolar configuration.

It will be appreciated that systems according to the invention may determine the emergence of an electrical event in a number of different ways, all of which are in accordance with the claimed invention. Where impedance is measured in first and second electrical pathways, as described above, a change may be detected when the impedance of the first electrical pathway increases or decreases relative to the second. Conversely, a change may occur when the impedance of the second electrical pathway increases or decreases relative to the first. In these scenarios, the impedance of one electrical pathway may increase in proportion to a decrease in the impedance of the other. For example, the impedance of the first electrical pathway may increase by some percentage, or by some absolute value, coinciding with a decrease of said percentage, or said absolute value, in the impedance of the second electrical pathway. Overall, the impedance of the circuit may not change, but the change in relative impedance between one pathway and another may indicate an electrical event in one or other electrical pathway. Of course, the change in impedance of one electrical pathway may not coincide with a corresponding change in the other. For example, the impedance of one electrical pathway may increase by some percentage, or by some absolute value, coinciding with the impedance of the other electrical pathway remaining the same, or decreasing by a greater or lesser proportion than the increase in the impedance of the first pathway, or even increasing by a greater or lesser proportion than the increase in the impedance of the first pathway. In all of these cases, the impedance of the first electrical pathway increases or decreases relative to the second (and vice versa), and the change may be indicative of an electrical event such as an electrical discontinuity or short circuit. By way of example, a difference (or relative change) in impedance between the electrical pathways may be indicative of an electrical event where the difference is between 20%-100%, 30%-90%, 40%-80%, 50%-70% or 50%-65%. A difference in impedance between the electrical pathways may be indicative of an electrical event where the difference is above 20%, 30%, 40%, 50%, 65%, 80%, 90%, or 100%. It is appreciated however, that these values are purely exemplary, and that others fall within the scope of the invention.

Alternatively (or in addition), a change may be detected when the overall impedance in both the first and the second electrical pathways increases or decreases. The increase or decrease in the impedance of the first electrical pathway may be identical to the increase or decrease in the impedance of the second electrical pathway, or it may be different. In any event, in all of these cases, the overall impedance increases or decreases, and the change may be indicative of an electrical event such as an electrical discontinuity or snort circuit. Of course, a combination of the changes described above may also be indicative of change. For example, a change may be determined based upon some combination of both a) an increase or decrease in impedance of one electrical pathway, together with b) an increase or decrease in the difference in impedance between the first and second electrical pathways.

A change may be detected upon an increase or decrease (be it the in absolute value of impedance of one electrical pathway or both electrical pathways, or in a percentage change, or in the difference between the first and second electrical pathways) exceeding a threshold value. The threshold value may be a predetermined threshold, which may be calculated from first principles or observed empirically.

Although two electrical pathways are described above, there may be more or fewer electrical pathways, such as one, three or more electrical pathways; the principles explained above remain the same. A higher number of electrical pathways may provide higher accuracy and/or resolution of the electrical defect detection owing to the additional information available from the additional electrical pathways.

Once a change is detected that is indicative of an electrical fault such as an electrical discontinuity, a short circuit, or microfracture, a number of actions are possible. For example, the action may be to cease the stimulation signal in one or both electrodes or electrode arrays, in particular in accordance with the nature of the detected change. For example, if an electrical discontinuity is determined because the impedance of the first electrical pathway increases by, for example, 75% whereas the impedance of the second electrical pathway stays the same, the system may cease the stimulation signal to the electrodes in the first electrical pathway but not the second. Alternatively, the system may cease the stimulation signal to the electrodes in both electrical pathways (i.e turn the implantable system off).

Rather than cease the stimulation signal in the electrodes of one or both electrical pathways, the action may be to switch operation of the system from bipolar stimulation to monopolar stimulation; or from tripolar stimulation to bipolar or monopolar stimulation. Again, this action has the effect of ceasing the delivery of the stimulation signal to the defective electrodes or electrode arrays.

A third alternative, the action may be to simply change stimulation signal, for example to raise or lower the current amplitude (or any other signal parameter) of the signal being delivered via electrodes or electrode arrays of the first or second pathways. In all three cases, but in particular in the final case where stimulation continues to be delivered, the action may include continuing to monitor impedance, or even increasing the frequency with which impedance measurements are taken.

Of course, whilst the system may be configured in such a way as to effect the aforementioned change automatically upon sensing a change indicative of a defect, in the alternative (or in addition), the system may be configured to transmit an alert to a remote, preferably external device so as to allow a user or a healthcare professional to change the operation of the system.

1-29. (canceled)

30. A system for applying electrical stimulation to a nerve, comprising:
an implantable housing comprising a signal generator;
an implantable neural interface for being in signaling contact with one or more nerves, the implantable neural interface comprising at least a first electrode and a second electrode electrically coupled to the signal generator for applying a signal generated by the signal generator to the one or more nerves;
an impedance measuring circuit electrically coupled to the first electrode and to the second electrode of the implantable neural interface and configured to measure an impedance of an electrical pathway therebetween; and
processing circuitry configured to periodically cause the impedance measuring circuit to measure impedances, the processing circuitry configured to determine a change in measured impedance that is indicative of an electrical event in the electrical pathway comprising one or both of the first electrode and the second electrode.

31. The system of claim 30, wherein the housing further comprises an electrically conductive portion for contacting tissue, in use, wherein the impedance measuring circuit is electrically coupled to the electrically conductive portion of the implantable housing to sense an impedance of an electrical pathway between the electrically conductive portion of the implantable housing and one or both of the first electrode and the second electrode.

32. The system of claim 30, further comprising:
a communication subsystem configured to couple to a remote device; and
a control system operatively coupled to the signal generator and configured to cause the communication subsystem to transmit an alert to the remote device in response to a determination by the processing circuitry that the change in measured impedance is indicative of an electrical event.

33. The system of claim 30, further comprising:
a control system operatively coupled to the signal generator and configured to modify the signal generated by the generator in response to a determination by the processing circuitry that the change in measured impedance is indicative of an electrical event.

34. The system of claim 31, wherein the signal generator is configured to generate the signal in either a first mode, wherein the signal is applied between the first electrode and the second electrode, or in a second mode, wherein the signal is applied between the electrically conductive portion of the implantable housing and either the first electrode or the second electrode.

35. The system of claim 34, further comprising:
a control system operatively coupled to the signal generator and configured to cause the signal generator to switch from generating the signal in the first mode to generating the signal in the second mode in response to a determination by the processing circuitry that the change in measured impedance is indicative of an electrical event.

36. The system of claim 35, wherein the control system is further configured to modify the signal generated by the generator in response to the determination by the processing circuitry that the change in measured impedance is indicative of an electrical event.

37. A system for applying electrical stimulation to a nerve, comprising:
an implantable housing comprising a signal generator and one or more transducers for producing corresponding electrical fields based on the signals generated by the signal generator;
an implantable neural interface for being in signaling contact with one or more nerves, the implantable neural interface comprising one more transducers for producing a signal based on the electrical fields generated by the one or more transducers of the implantable housing; at least a first electrode and a second electrode electrically coupled to the one or more transducers for applying the signal produced by the one or more transducers to the one or more nerves;
the implantable housing comprising an electrical field sensing circuit configured to sense a first electrical field produced by the first electrode and a second electrical field produced by the second electrode of the implantable neural interface; and
processing circuitry configured to periodically cause the electrical field sensing circuit to sense electrical fields, the processing circuitry configured to determine a change in the sensed electrical fields that is indicative of an electrical event in one or both of the first electrode and the second electrode.

38. The system of claim 37, further comprising:
a communication subsystem configured to couple to a remote device; and
a control system operatively coupled to the signal generator and configured to cause the communication subsystem to transmit an alert to the remote device in response to a determination by the processing circuitry that the change in sensed electrical fields is indicative of an electrical event.

39. The system of claim 37, further comprising:
a control system operatively coupled to the signal generator and configured to modify the signal generated by the generator in response to a determination by the processing circuitry that the change in sensed electrical fields is indicative of an electrical event.

40. The system of claim 30, wherein the processing circuitry is configured to generate data corresponding to said measured impedances or said sensed electrical fields over time and to determine the change in measured impedance from the generated data.

41. The system of claim 30, wherein the electrical event is one or more of a short circuit, and an electrical discontinuity.

42. The system of claim 30, wherein the processing circuitry is configured to detect a difference in measured impedance between a first electrical pathway and a second electrical pathway.

43. The system of claim 42, wherein the processing circuitry is configured to determine an electrical event when the difference in measured impedance between the first electrical pathway and the second electrical pathway is between 20%-100%, optionally between 30%-90%, optionally between 40%-80%, optionally between 50%-70%.

44. The system of claim 30, wherein the processing circuitry is configured to calculate a baseline value of impedance from one or more of said measured impedances or said sensed electrical fields, further wherein the processing circuitry is configured to determine the change in measured impedance by comparing the measured impedance with the baseline value of impedance.

45. A method of determining an electrical event in an electrical pathway comprising:
a first electrode of an implantable neural interface, in a system for applying electrical stimulation to a nerve, the method comprising:
periodically sensing an impedance in at least an electrical pathway between the first electrode and either a second electrode or an electrically conductive portion of a housing of the system; and
determining an electrical event in the electrical pathway comprising the first electrode by identifying a change in the sensed impedance.

46. The method of claim 45, wherein the step of determining an electrical event comprises identifying a change in the rolling average impedance of the measured impedances.

47. The method of claim 45, wherein the step of determining an electrical event comprises:
calculating a baseline value of impedance from one or more of said sensed impedances, further
wherein the step of determining an electrical event comprises identifying a change in the sensed impedance by comparing the sensed impedance with the baseline value of impedance.

48. The method of claim 45, wherein the step of periodically sensing an impedance further comprises periodically sensing a first impedance in a first electrical pathway between the electrically conductive portion of the implantable housing and the first array of electrodes and periodically sensing a second impedance in a second electrical pathway between the electrically conductive portion of the implantable housing and the second array of electrodes; and wherein
the step of determining an electrical event in the first array of electrodes comprises determining that the difference between the first impedance and the second impedance exceeds a predetermined threshold.

49. The method of claim 45, wherein the step of periodically sensing an impedance further comprises periodically sensing a first impedance in a first electrical pathway between the electrically conductive portion of the implantable housing and the second array of electrodes and periodically sensing a second impedance in a second electrical pathway between the first array of electrodes and the second array of electrodes; and wherein:
the step of determining an electrical event in the first array of electrodes comprises determining that the second impedance exceeds a predetermined threshold.

50. The method of claim 45, further comprising a step of identifying stimulation artefact in an electrocardiogram signal concurring with application of electrical stimulation.
